(19) United States
(12) Patent Application Publication
(10) Pub. No.: US 2004/0035186 A1
(43) Pub. Date: Feb. 26, 2004

(54) Interface between a collector and a package

(76) Inventors: William C. Allen, Colorado Springs, CO (US); Leighton Ige, Honolulu, HI (US)

Correspondence Address: Blaney Harper, Esq., Jones Day, 51 Louisiana Avenue, N.W., Washington, DC, 20001-2113, US

(21) Appl. No.: 10/406,842

(22) Filed: Apr. 4, 2003

Related U.S. Application Data
(63) Continuation-in-part of application No. 10/224,719, filed on Aug. 21, 2002, now Pat. No. 6,651,520.
(63) Continuation-in-part of application No. 10/224,688, filed on Aug. 21, 2002.

(51) Int. Cl. G01N 1/24; G01N 33/22; G01N 33/15

(52) U.S. Cl. 73/31.03; 73/31.07; 73/863.23; 73/863.81

(57) ABSTRACT

The present invention is a novel collection device that allows constituents of air to collect on a test strip included within the collection device. Specifically, the collector allows a removable test strip to be inserted in the collector while an air sample passes therethrough. Thereafter, the test strip is removed from the collector and analyzed to determine the constituents of air that passed through the test strip and collector. When using the collector to inspect for trace amounts of explosives within a package, the collector is connected to a package-collector interface because the shape of the package-collector interface corresponds to that of the collector. An air sample is extracted from the interior of the package and through both the interface and the collector, including the test strip. As the air is extracted, the constituents of air collect on the test strip. After collecting the constituents, the test strip is removed from the collector and inserted into a testing unit capable of detecting trace amounts of explosives. The testing unit subsequently analyzes the constituents and determines whether any of them are explosive.

### CROSS-REFERENCE TO RELATED APPLICATIONS

This is a continuation-in-part of application Ser. No. 10/224,688 filed Aug. 21, 2002 and application Ser. No. 10/224,710 filed Aug. 21, 2002.

### TECHNICAL FIELD

This invention relates to the field of testing for explosives and more particularly, a collection device that collects the constituents of air extracted from the interior of items, such as baggage and packages, wherein the collection device is subsequently tested to determine whether the items contain explosives.

### BACKGROUND

Aircraft and particularly, those operated by passenger airlines, are considered terrorist targets. Thus, aviation security is essential to the safety of airline passengers. One attempt to minimize the possibility of a terrorist attack includes screening all baggage (i.e., luggage) that enters the aircraft. Specifically, both carryon and checked baggage are screened for bombs and/or explosives.

Some of the current methods used to screen carry-on and checked baggage include manual inspection, X-ray, and trace detection. Manual inspection is invasive and often time consuming. Utilizing X-ray equipment is a non-invasive procedure. However, that method requires the operator of the X-ray equipment to promptly recognize the particular shape of the explosive device.

Trace detection is also less intrusive than manual inspection. Trace detection generally means testing for trace (i.e., small) amounts of explosives. If trace amounts of explosives are detected, then such a result may be indicative that a piece of baggage contains explosives. One current trace detection technique includes swiping the exterior of a piece of baggage with a test strip and subsequently placing the test strip in a testing unit capable of detecting trace amounts of explosives.

One testing unit capable of detecting trace amounts of explosives is an EGIS Explosives Detection System sold by Thermo Detection in Chelmsford, Mass. The EGIS Explosives Detection System is based on analyzing a test strip, which is also referred to as a ticket. FIG. 1 illustrates such a test strip 100, which comprises two folded portions 102, 104 constructed of relatively firm sheet stock (i.e., paper) or thin cardboard. One of the folded portions 102 comprises an aperture (i.e., hole or opening), which is overlapped (i.e., covered) by a porous material, such as cloth or a teflon-coated cloth. That is, the porous material adheres to one side of the folded portion 102, and preferably the side that is closer to the other folded portion 104 when the test strip 100 is folded.

The test strip 100 is currently used in a baggage-screening process at an airport. For example, a security person who screens baggage folds one portion 104 of the test strip 100 such that the portion 104 is adjacent the other portion 102 and the porous material 106. The security person will thereafter swipe the portion 102, including the porous material 106, of test strip 100 over the exterior of a piece of baggage. Specifically, the security officer, using a finger, will depress the opposite side of the portion 104 aligned with the porous material 106 while swiping the baggage with the test strip 100, thereby applying pressure to the porous material as it contacts the baggage. As the test strip 100 passes over the exterior of the baggage, various particles and molecules adhere to the porous material.

Referring to FIGS. 2 and 2A, after the security person swipes the baggage with the test strip 100, the security person places the test strip 100 in the testing unit 200, such as Thermo Detection's EGIS Explosives Detection System, capable of detecting trace amounts of explosives. Specifically, the security person inserts the test strip into the testing unit 200 such that portion 102 and porous material 106 is first inserted into the testing unit. Upon operating the testing unit with the test strip placed within the unit, the testing unit indicates whether the test strip includes trace amounts of explosives, thereby indicating that the exterior of the baggage includes trace amounts of explosives.

Although this method of using test strips assists in detecting explosive particles and molecules on the exterior of baggage, this current trace detection method does not determine whether trace amounts of explosives are present within the interior of the baggage. That is, swiping the exterior of baggage using a test strip does not allow the security person to inspect the interior of the baggage without opening it. Furthermore, manually handling of the test strips such as touching the porous material with a forefinger may lead to the contamination of the test strips. What is needed is a non-invasive method of inspecting the interior of the baggage while minimizing the likelihood of contamination.

### OBJECTS OF THE INVENTION

It is an object of the invention to determine whether explosives are contained within a piece of baggage.

It is another object of the invention to determine whether explosives are contained within a piece of baggage via a noninvasive procedure.

It is another object of the invention to create a new type of collector for collecting trace amounts of explosives.

It is another object of the invention to create a collector that incorporates an existing test strip.

If is a further object of the invention to use the test strip within a collector in order to collect the constituents of air from within the interior of a piece of baggage.

If is a further object of the invention to utilize the test strip in determining whether explosives are contained within a piece of baggage.

It is even a further object of the invention to create an interface between the baggage and the collector.

### SUMMARY OF THE INVENTION

The present invention is a collector that includes a test strip capable of capturing constituents of air as air passes through the collector. The collector is designed such that the test strip can easily be inserted into and removed from the collector. Because the collector is envisioned to be used in a method of extracting air from the interior of a piece of baggage to test for the presence of explosives within the baggage, the test strip is inserted into the collector prior to extracting air through the collector and thereafter removed from the collector and inserted an explosives testing unit.

Utilizing the known test strip or a modified version of the test strip allows the method of the present invention to utilize existing testing equipment. That is, the collector of the present invention incorporates the test strip into an air extraction process compared to using the test strip in a physical swiping process discussed above in the background section. By designing the collector of the present invention to include a test strip, a modified version of the test strip can be used by the same testing unit used in the physical swiping process. But rather than testing for the presence of trace amounts of explosives on the exterior of the baggage, inserting the test strip into the testing unit, and operating the testing unit will indicate whether the interior of the baggage includes trace amounts of explosives.

The collector is connected to an interface between it and a piece of baggage or a package. For the purposes of this invention, such an interface shall be referred to as a baggage-collector interface or a package-collector interface, respectively. The baggage-collector interface is able be secured to the opening in the baggage, such as an opening created by a zipper. After the baggage-collector interface is clamped to the teeth of the zipper and the zipper is closed, the collector is firmly and securely inserted into the baggage-collector interface because the shape of such interface matingly engages the collector. The baggage-collector interface, therefore, provides a securing mechanism through which an air sample may be extracted from the interior of the baggage and through the collector.

The baggage-collector interface provides a convenient entry point from which to extract air from the interior of baggage. Moreover, the shape of such interface corresponds to that of the collector. Thus, an air sample passes through both the interface and the collector. Because the air sample passes through the collector and particularly the porous material of the test strip, the constituents of air collect thereon. That is, assuming explosives reside in the baggage, constituents of the air will include trace amounts of such explosives, which will gather on the porous material of the test strip in the collector as the air sample is extracted from the baggage. For the purpose of this invention, the term “constituents” shall mean both particles in the air and vapor molecules. Thereafter, the test strip can be placed in a testing unit capable of detecting trace amounts of explosives and analyzed to determine the nature of the constituents collected on the porous material to determine whether any of the constituents are explosive.

Similar to using a baggage-collector interface and a collector to extract from the interior of a piece of baggage, a package-collector interface and the same collector could be used to extract air from the interior of a package. A package may include a pouch or box used by courier services, such as FedEx®, UPS® or Airborne Express®. Although the same collector and a similar method of extracting air from the interior of the package is used to extract air from within the package, a different interface would likely be used. That is, the interface used to extract air from the interior of a package would be a package-collector interface, wherein the package-collector interface is attached to or embedded within the package.

The foregoing features and advantages of the present invention will become more apparent in light of the following detailed description of exemplary embodiments thereof as illustrated in the accompanying drawings.

### BRIEF DESCRIPTION OF DRAWINGS

FIG. 1 is a perspective of an existing test strip 100, which is also referred to as a ticket.

FIGS. 2 is an illustration of a testing unit 200, such as the EGIS Explosives Detection System sold by Thermo Detection, into which the test strip 100 of FIG. 1 is inserted and analyzed.

FIG. 2a is an enlarged view of a portion of FIG. 2 illustrating the insertion of the test strip 100 into the testing unit 200.

FIG. 3 is a flow diagram of one embodiment of the method of the present invention for testing the air within the interior of a piece of baggage.

FIG. 4 illustrates a baggage-collector interface 400 being inserted into a piece of baggage 404 and particularly, into the zipper 402 of the baggage 404.

FIGS. 5A and 5B are enlarged views of the baggage-collector interface 400 inserted within the baggage's zipper 402 as seen in FIG. 4.

FIG. 6 is a perspective view of one embodiment of the baggage-collector interface 400 of the present invention.

FIG. 7 is a top view of one embodiment of the baggage-collector interface 400 of the present invention.

FIG. 8 is a side view of one embodiment of the baggage-collector interface 400 of the present invention.

FIG. 9 is an end view of one embodiment of the baggage-collector interface 400 of the present invention, wherein such interface comprises means for gripping the zipper 402.

FIG. 9A is an enlarged view of the means for gripping the zipper 402.

FIG. 10 is a perspective view of one embodiment of the collector 1000 of the present invention.

FIG. 11 is a top view of one embodiment of the collector 1000 of the present invention.

FIG. 12 is an end view of one embodiment of the collector 1000 of the present invention.

FIG. 13 is a side view of one embodiment of the collector 1000 of the present invention.

FIG. 14 is a perspective view of an alternate embodiment of the collector 1000′ of the present invention.

FIG. 15 is a top view of an alternate embodiment of the collector 1000′ of the present invention.

FIG. 16 is an end view of an alternate embodiment of the collector 1000′ of the present invention.

FIG. 17 is a side view of an alternate embodiment of the collector 1000′ of the present invention.

FIG. 18 is a side view of one embodiment of the collection system 1800 of the present invention comprising a collector 1000 matingly engaged with a baggage-collector interface 400.

FIG. 19 illustrates the insertion of the collection system 1800, particularly the baggage-collector interface 400, into a piece of baggage and extracting a sample of air from within the baggage by vacuuming the air through the interface 400 and collector 1000.

FIG. 19A is an enlarged view of the collection system 1800 inserted within a piece of baggage and connected to a vacuum hose used to extract air from within the baggage.

FIG. 20 is a flow diagram of one embodiment of the method of the present invention for testing the air within the interior of a package.

FIG. 21 is a perspective view of a package 2100 comprising a package-collector interface 2200 of the present invention.

FIG. 22 is a perspective view of one embodiment of the package-collector interface 2200 of the present invention.

FIG. 23 is a top view of one embodiment of the package-collector interface 2200 of the present invention.

FIG. 24 is an side view of one embodiment of the collector 1000 of the present invention.

### DESCRIPTION OF THE PREFERRED EMBODIMENTS

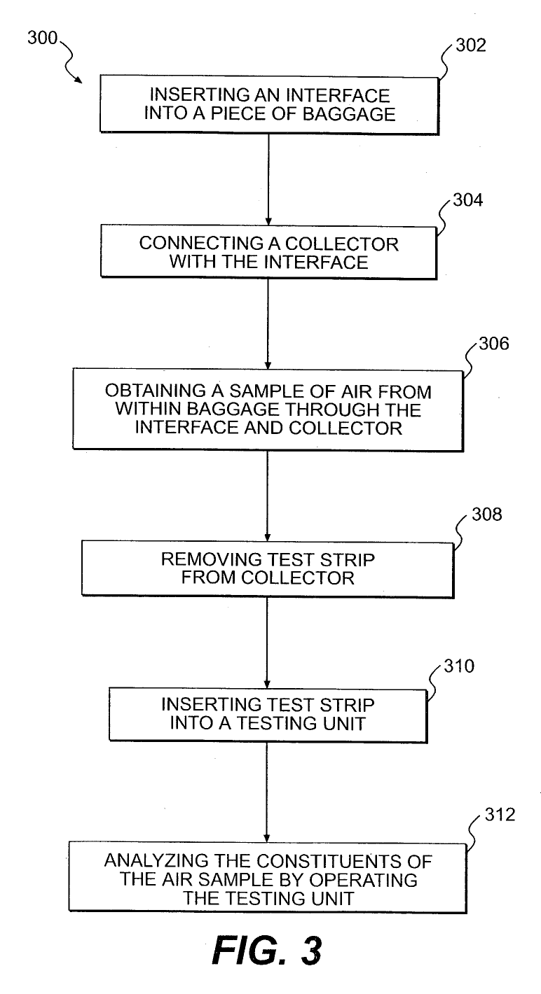

Referring to FIG. 3, there is shown a flow diagram of one embodiment of the method of the present invention. The method of the present invention is directed towards analyzing the air from the interior of a piece of baggage to determine whether explosives are included therein. Although the steps of the invention are listed in a particular order in FIG. 3 and explained in such order, it is not necessary that the invention be practiced in that exact order. Moreover, additional steps may be added to those illustrated in FIG. 3 and still be within the scope of this invention.

The steps shown in FIG. 3 include: inserting a baggage-collector interface into a piece of baggage 302; connecting a collector, which includes a test strip, with the baggage-collector interface 304; drawing a sample of air from within the baggage by vacuuming the air through the baggage-collector interface and the collector (including the test strip) 306, thereby allowing constituents (i.e., particles or vapors) of air to adhere to and collect on the test strip in the collector; removing the test strip from the collector 308; inserting the test strip into a testing unit capable of detecting a trace amount of an explosive 310; and analyzing the constituents of the air sample by operating the testing unit 312.

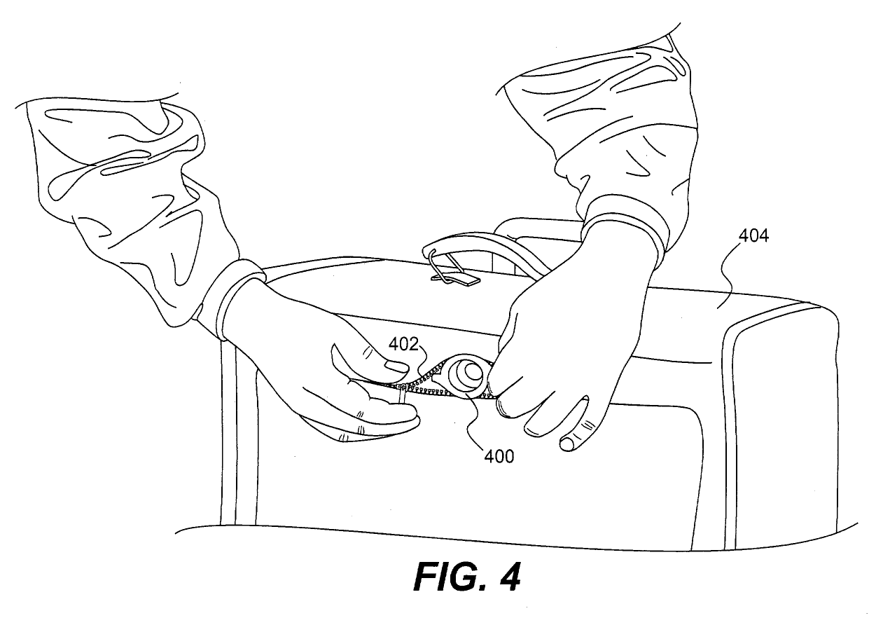

FIG. 4 illustrates one embodiment of step 302—the step of inserting a baggage-collector interface 400 into a piece of baggage 404. Specifically, FIG. 4 illustrates an individual inserting a baggage-collector interface 400 into the zipper 402 of a piece of baggage 404. Although FIG. 4 illustrates the baggage-collector interface 400 located within the zipper 400 of the baggage 404, it shall be understood that the scope of the present invention includes temporarily or permanently placing the baggage-collector interface 400 within the body of the baggage. For example, it may be desirable to retrofit luggage to include a baggage-collector interface or install a baggage-collector interface within the baggage during manufacturing of the baggage.

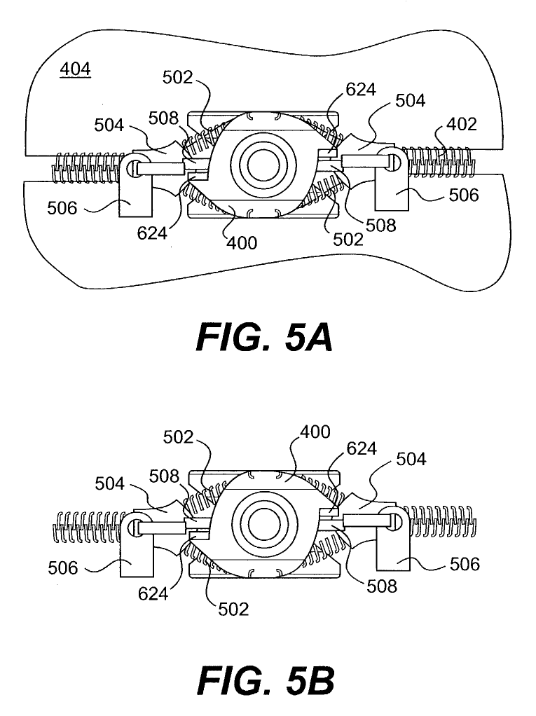

FIGS. 5A and 5B are enlarged views of the baggage-collector interface 400 inserted within the zipper 402 of the baggage 404. Those figures illustrate that the zipper 402 includes two rows of teeth 502 and two sliders 504, which further include pull tabs 506. And when the baggage-collector interface 400 is inserted into the zipper 402, the baggage-collector interface 400 grips at least a portion of each row of teeth 502.

Once the baggage-collector interface 400 is gripping the zipper's teeth 502, it may be preferable to close the zipper 402. Closing the zipper may include the step of sliding one or both of the sliders 504, via pulling the pull tabs 506, toward the baggage-collector interface 400 until the slider(s) 504 are adjacent and/or abut the baggage-collector interface 400. Some sliders 504 may include locking rings 508, which allow the sliders 504 to be locked together, thereby preventing someone from opening the zipper 402 without first removing a lock. As will be discussed below, it may be preferable for the baggage-collector interface 400 to include corresponding (i.e., complementary) locking rings 624. If the baggage-collector interface 400 includes locking rings 624, it may be desirable to slide the slider(s) 504 toward the baggage-collector interface 400 until the openings within the locking rings 624 of the sliders 506 align with the openings in the locking rings 504 of the baggage-collector interface. Once the locking rings 504, 624 are aligned, it would be possible to secure the baggage-collector interface 400 to the sliders 504 by passing a lock or other type of fastening means through the holes within the locking rings 504, 624.

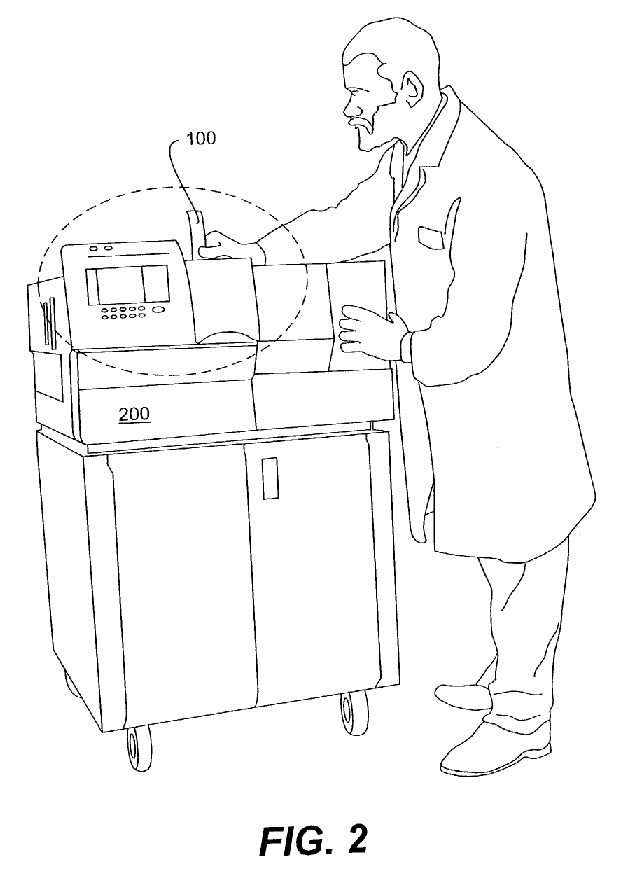

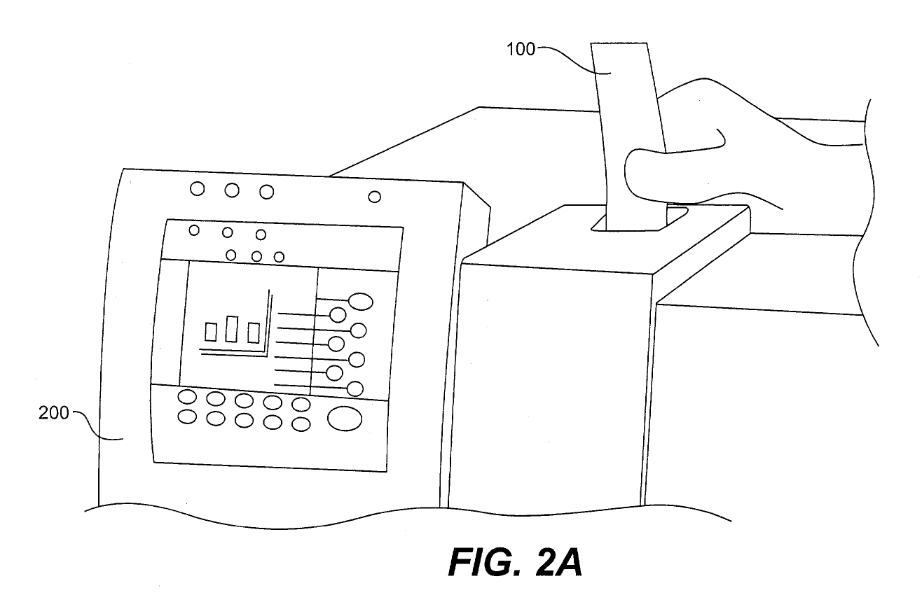

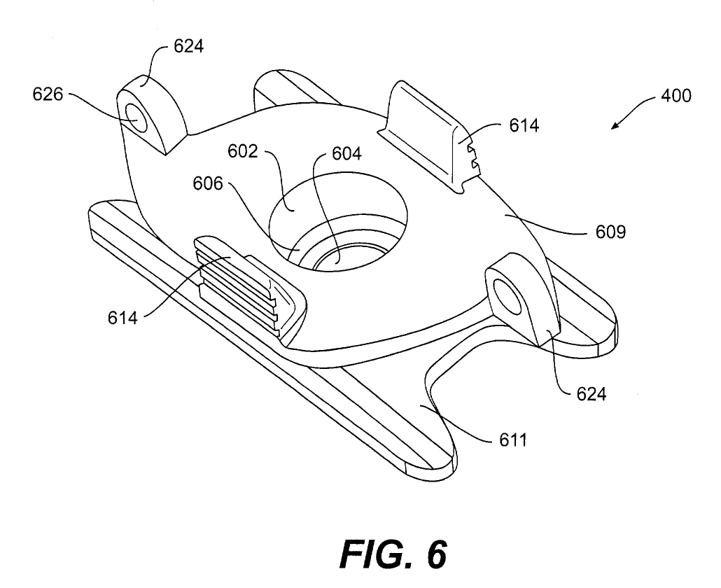

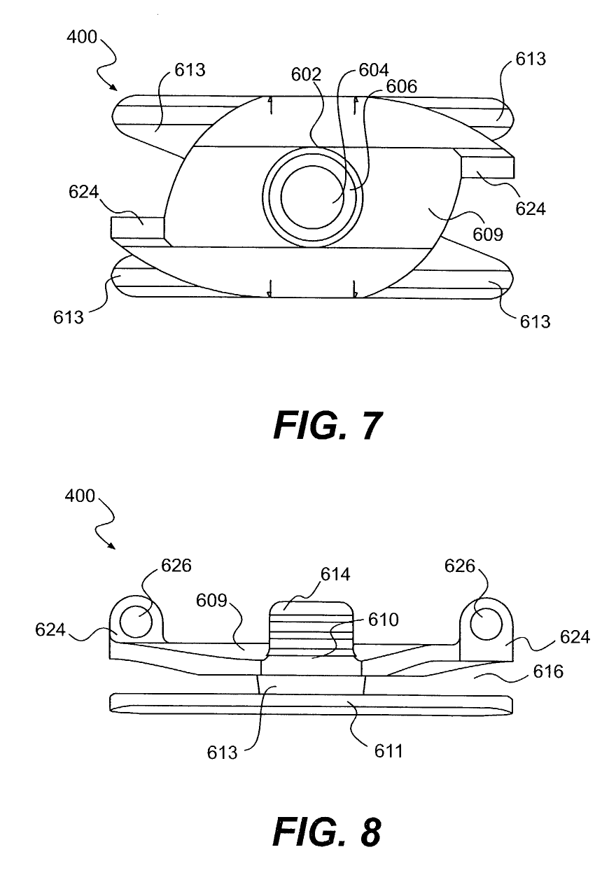

FIGS. 6 to 9 illustrate one embodiment of the baggage-collector interface 400 of the present invention. That embodiment includes a recess 602 which has a shape that corresponds to the shape of the exterior of the collector 1000 discussed in detail below, thereby allowing the collector 1000 and the baggage-collector interface 400 to interconnect. As illustrated in FIGS. 2A and 2B, the recess 602 has a cylindrical shape. This is due to the fact that, the perimeter (i.e., circumference) of the portion of the collector 1000 which is inserted into the baggage collector interface 400 is circular. Because the shape of the perimeter of the recess 602 corresponds to the shape of the collector 1000, the perimeter of the recess 602 is shown as circular.

The present invention, however, shall not be limited to using collectors that have a circular exterior or cylindrical shape. Rather, the scope of this invention shall also apply to collectors that have other shapes, such as any type of polygon or non-linear shape. What is important is that the perimetrical shape of the recess corresponds to the exterior shape of the collector such that when the collector is inserted into the recess of the baggage-collector interface, the recess matingly engages the collector and the two articles fit properly (i.e., firmly and securely) together.

Included within the recess 602 is a vent 604 that allows air to pass from one side of the baggage-collector interface 400 to the other. When the baggage-collector interface 400 is inserted within the baggage's zipper 402, the air within the baggage may pass through the baggage-collector interface 400 to the outside of the baggage. Thus, the vent 604 provides a means for air to travel through the baggage-collector interface 400 and into the collector 1000. Accordingly, it is preferable that the vent 604 align with an opening of the collector 1000.

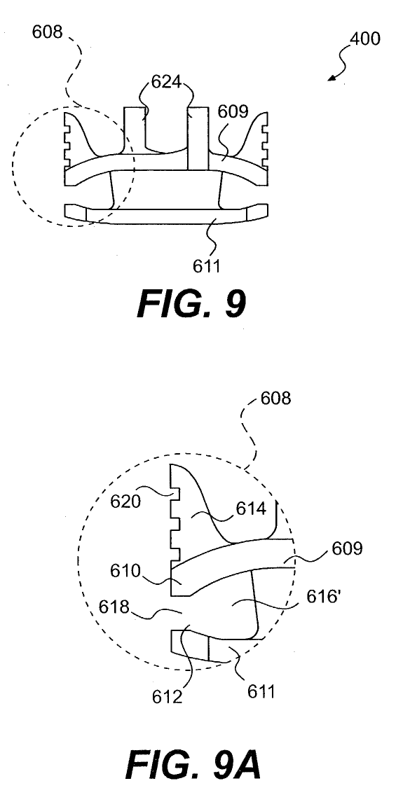

Continuing to refer to FIGS. 6 to 9, including FIG. 9A, the baggage-collector interface 400 includes top and bottom securing members 609, 611 connected to one another via an interface tube 613. It is the interior of the interface tube 613 that forms the recess in the baggage-collector interface 400. The top and bottom securing members 609, 611 preferably have a circular shape when viewed from the top or the bottom of the baggage-collector interface 400. When the baggage-collector interface 400 is viewed from the side, a gap 616-created by the interface tube 613-exists between the top and bottom securing members 609, 611. Thus, when the baggage-collector interface 400 is inserted into the opening within the piece of baggage, the baggage-collector interface 400 can be turned (i.e., rotated), and as it turns, the baggage-collector interface 400 clasps the baggage. That is, when the baggage-collector interface 400 is inserted into the baggage, the baggage, and preferably the zipper, will be in the gap 616 between the top and bottom securing members 609, 611 created by the interface tube 613.

The top and bottom securing members 609, 611 provide the baggage-collector interface 400 means for gripping the opening of the baggage. Preferably, the baggage-collector interface 400 is inserted into the zipper 402 of a baggage 404 as shown in FIGS. 4 and 5. Thus, the top and bottom securing members 609, 611 provide the baggage-collector interface 40 with means for gripping a portion of each row of teeth 502 of the zipper 402.

It may also be preferable for the bottom securing member 611 to include wing portions 613 on one or both sides of the baggage-collector interface 400. As illustrated in FIGS. 5A and 5B, the wing portions 613 of the bottom securing member 611 will be inside the baggage when the baggage-collector interface 400 is inserted into the baggage. The wing portions 613 provides the baggage-collector interface 400 with added stability when it is inserted into the baggage.

In an alternate embodiment of the present invention, it may be preferable for the baggage-preconcentrator interface 400 to include two clamps 608, which can provide additional means for gripping the opening of the baggage. As illustrated in FIG. 9A, the top securing member 609 may have a top lip portion 610, and the bottom securing member 611 may have a bottom lip portion 612. It is also preferable that the shape of the top lip 610 create a gap 616′ between it and the bottom lip 612. Although it is not necessary, the gap 616′ may also include an opening 618 such that the top and bottom lips 610, 612 closely approximate but do not contact one another. The shape of the gap 616′ illustrated in FIG. 9A allows the clamps 608 of the baggage-collector interface 400 to grip the teeth of a zipper. As mentioned above, however, the baggage-collector interface 400 may be inserted into another portion of the baggage, such as the body. Accordingly, the shape of the gap and opening may change.

For example, the top and bottom lips 610, 612 may initially contact one another. Assuming the top and bottom lips 610, 612 initially contact one another, the baggage-collector interface 400 may include a means for opening the lips so that the baggage-collector interface 400 can be inserted into the baggage. Similarly, if the baggage-collector interface 400 has a gap and opening analogous to those illustrated in FIG. 9A, a means for enlarging the opening and/or gap may be included in order to insert the baggage-collector interface 400 into the baggage. Thus, it may be preferable for the clamp 608 to include a lever 614 that is connected to the top lip 610, such that when the lever 614 is moved (i.e., pressed) toward the center of the baggage-collector interface 400, the size of the opening 618 will increase. Further assuming that the clamps 608 are diametrically opposed to one another, it may be preferable for the lever 614 to include grooves 620 (i.e., channels, serrations, etc.) so that an individual is able to have a firm grasp of the levers 614 when the individual simultaneously squeezes the levers 614 toward one another with his hand (e.g., thumb and forefinger).

As mentioned above, some zippers include sliders 504 that have means for allowing a lock to secure (i.e., fasten) two sliders 504 together. Thus, it may be preferable for the baggage-collector interface 400 to include means for securing itself to two sliders 504 when such sliders abut the baggage-collector interface 400. One such means includes locking rings 624 connected to the baggage-collector interface 400. Each locking ring 624 includes an opening 626 that can be aligned with an opening in the baggage's locking ring 508. Upon placing a lock or some other fastening means through both the baggage-collector interface and baggage locking rings 508, 624, the baggage-collector interface 400 will be secured to the zipper's slider 504. Although it may be preferable for the baggage-collector interface 400 to have two locking rings 624, it may be sufficient to have only one locking ring 624.

The baggage-collector interface 400 may be constructed of many different types of materials. It is preferable that the baggage-collector interface 400 be somewhat flexible, yet firm. Thus, it may be preferable to construct the baggage-collector interface 400 from plastic(s), such as polypropylene. One such method of constructing the baggage-collector interface 400 from polypropylene is an injection molding process.

After the baggage-collector interface 400 is securely inserted into the baggage 404, the collector 1000 is connected to the baggage-collector interface 400. This connection preferably occurs by inserting the collector 1000 into the recess 602 of the baggage-collector interface 400. As discussed above, the recess of the baggage-collector interface 400 and the exterior of the collector 1000 are designed to matingly engage such that upon inserting the collector 1000 into the recess, the collector 1000 and baggage-collector interface 400 fit firmly together.

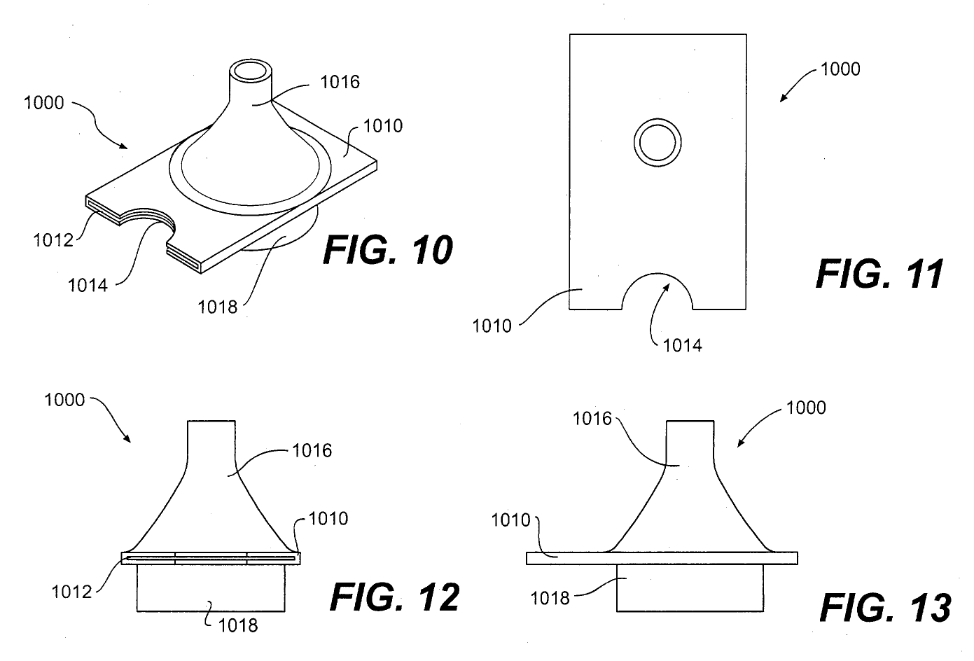

Referring to FIGS. 10-13, there is shown one embodiment of the collector 1000 of the present invention. The collector 1000 includes a slotted plate 1010. That is, the collector 1000 includes a plate 1010 that includes a slot 1012. The plate 1010 is slotted so that a test strip 100 may be inserted therein. In order to provide easy installation and removal of the test strip 100, the slotted plate is open at one end. That is, it is preferable for the slotted plate 1010 to be open at one end and closed at the other end. Furthermore, it is preferable that the slotted plate 1010 include a concave indentation 1014 at its open end, thereby allowing a person to easily as the test strip 100 when it is within the slot 1012 and remove it. Thus, the concave indentation 1014 is perpendicular to the slot.

Although not shown, the slotted plate 1010 includes an aperture (i.e., opening) parallel to the concave indentation 1014 and perpendicular to the slot 1012 in the plate 1010. Furthermore, as will be discussed in more detail below, the aperture is aligned with tubular portions 1016, 1018 of the collector 1000, thereby allowing air to pass through the slotted plate 1010 and through the collector 1000. That is, the test strip 100 is inserted into the slot 1012 such that the porous material 106 in the test strip 100 aligns with the aperture in the slotted plate 1010. Because the porous material 106 is aligned with the aperture, the porous material will collect (i.e., capture) constituents of air as the air passes through the collector.

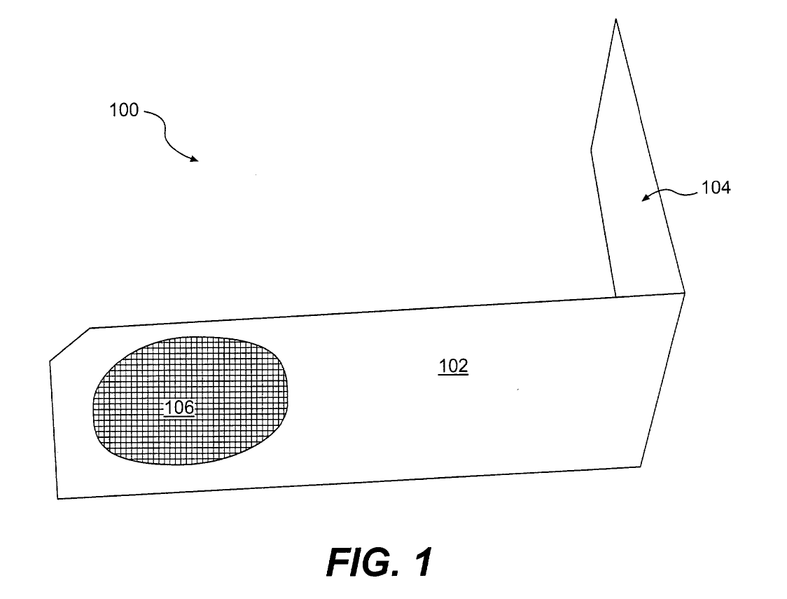

As mentioned above, it may be desirable to utilize a modified version of a known test strip 100. For example, it may be desirable to utilize a test strip comprising a porous material having a pore size larger than the pore size of the porous material of the test strip 100 illustrated in FIG. 1. That is, depending upon the size of the collector and the volumetric flow rate of air passing therethrough, the pore (i.e., mesh) size of the existing porous material may be too fine (i.e., small) thereby creating an undesirable pressure drop across the porous material. For example, the pore size of the teflon-coated cloth is smaller then the non-coated cloth. Therefore, it may be desirable to use the non-coated cloth or increase the pore size of any desirable type of porous material to decrease the pressure drop. Nevertheless, it is still desirable to maintain a relatively small pore size in order for the constituents of air to collect (i.e., gather) on the porous material as the air passes therethrough. Moreover, it may be desirable to utilize a test strip composed of only one portion rather than comprising two folded portions 102, 104 as illustrated in FIG. 1. That is, the folded portion 104 may be helpful when using the ticket 100 in a swiping test, but may be unnecessary when the ticket is inserted into the collector of the present invention.

The collector 1000 also includes two tubular portions 1016, 1018 extending from both sides of the slotted plate 1010 thereby providing a passageway through the collector 1000 and the slotted plate 1016. As illustrated in FIGS. 10, 12 and 13, tubular portion 1016 extends from the top of the slotted plate 1010, and tubular portion 1018 extends from the bottom of the slotted plate 1010. As will be discussed below, a hose will be inserted in or placed over tubular portion 1018 so that air may be extracted through the collector 1000. Tubular portion 1016, alternately, will be inserted into the recess 602 of the baggage-collector interface 400.

The tubular portion 1016 preferably has a conical shape. It is even more preferable that tubular portion 1016 have a funnel-type shape such that the larger end of the funnel extends from the slotted plate 1012 and the smaller end of the funnel is further from the slotted plate 1012. Utilizing such a funnel-type shape provides the desirable airflow through the collector. That is, as the air travels through the collector, and particularly the porous material in the test strip, a certain velocity and volumetric flow rate of air is necessary to overcome the pressure drop as the air passes through the porous material. Utilizing such a funnel-type shaped tube, in conjunction with the appropriately sized pores in the porous material, provides the desired flow-rate of air through the porous material.

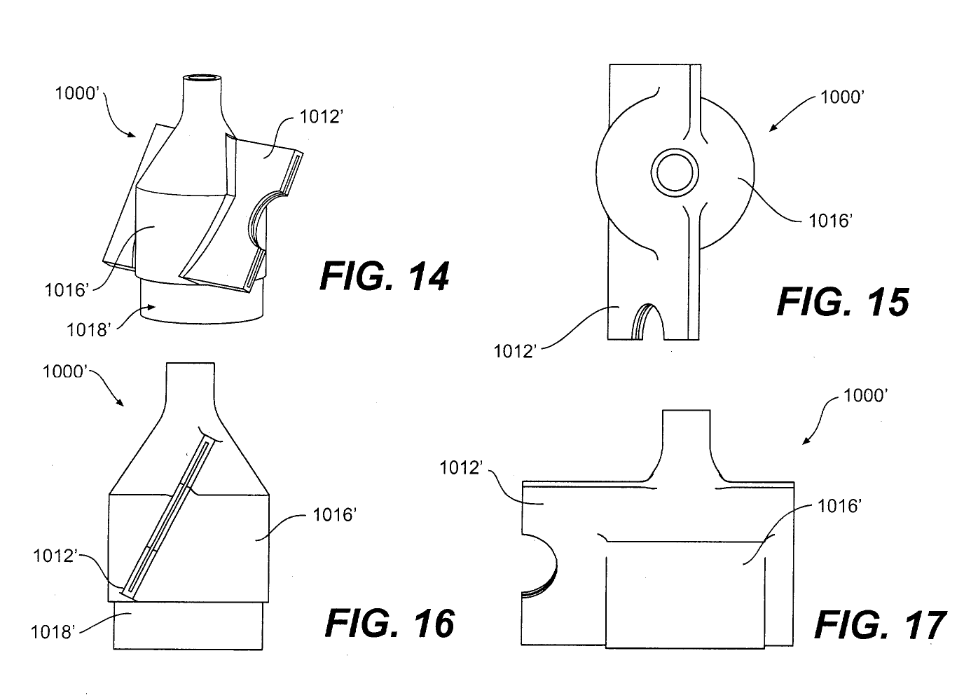

An alternate embodiment of the collector 1000 of the present invention is illustrated in FIGS. 14-17. The primary distinction between the embodiment of the collector 1000 illustrated in FIGS. 10-13 and the collector 1000 illustrated in FIGS. 14-17 is that the slotted plate 1012 in the initial embodiment separates tubular portions 1016, 1018 while the slotted plate 1012 in the alternate embodiment of the collector 1000 is included within tubular portion 1016. That is, in the embodiment of the collector 1000 illustrated in FIGS. 10-13, the slotted plate 1012 is perpendicular to the orifices of tubular portions 1016, 1018 and the embodiment of the collector 1000 illustrated in FIGS. 14-17, the slotted plate 1012 is at an angle acute to the orifices of tubular portions 1016, 1018. Placing the slotted plate 1012 at an angle acute to the orifices of the tubular portions 1016, 1018 increases the width of the slotted plate 1012, which in turn increases the overall surface area of the porous material 106 in the slotted plate 1012, thereby increasing the probability of capturing constituents of air on the porous material 106. Furthermore, although tubular portions 1016, 1018 are illustrated as two separate portions, it is possible that the exterior and/or interior orifice of the two tubular portions may be the same size. If so, the collector 1000 would appear to be constructed of one tubular portion.

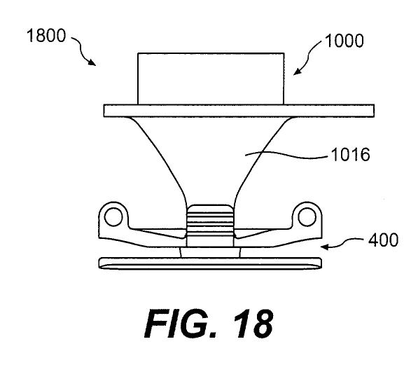

Referring to FIG. 18, assuming the embodiment of collector 1000 is used, the collector 1000 is connected to the baggage-collector interface 400 by inserting the collector 1000 into the baggage-collector interface 400. Tubular portion 1016 of the collector 1000 is designed to correspond to the recess 602 formed by the interface tube 613 in the baggage-collector interface 400, thereby allowing the collector 1000 and baggage-collector interface 4000 to matingly engage. Although the embodiment of the present invention is illustrated and described such that the baggage-collector interface 400 includes a recess 602 having a tubular portion 1016 which is inserted into the recess 602, it shall be understood that the baggage-collection interface 400 may include a tubular extension and the collector 1000 may include a recess into which the tubular extension of the baggage-collector interface may be inserted. That is, the items having the recess and the tubular portion may be reversed compared to the embodiment illustrated in FIG. 18.

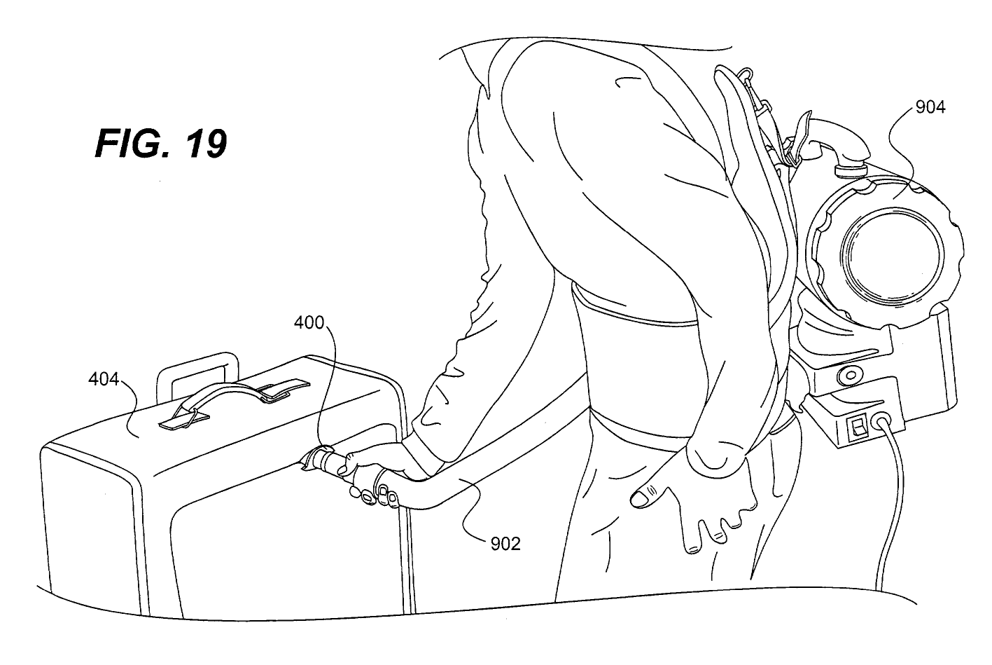

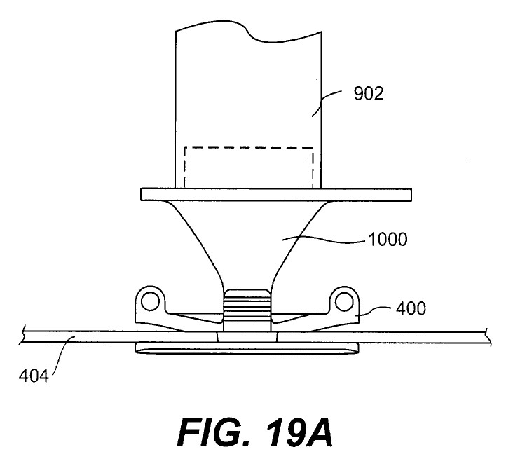

Referring to FIGS. 19 and 19A, after the collector 1000 is connected to baggage-collector interface 400, air is extracted from the interior of the piece of baggage 404 through the collection system 1200 (i.e., collector 1000 baggage-collector interface 400). Specifically, air is extracted from the interior of the piece of baggage through the vent 604 in the baggage-collector interface 400 and through the passageway in the collector 1000 created by tubular portion(s) 1016, 1018 and the porous material 106 of the test strip 102 inserted in the slotted plate of the collector 1000 because the vent, passageway, porous material (and aperture covered by the porous material) align with one another when the collector 1000 is inserted into the baggage-collector interface 400.

One means for extracting air from inside of the baggage includes securing (i.e., connecting) the collector 110 to one end of a hose 902, the other end of which is connected to a vacuum 904, as illustrated in FIG. 19. When the vacuum 904 is turned “ON”, a pressure differential across the collector 110 is created and air is extracted from the interior of the baggage 404 and through collection system 1000. For a typically sized piece of baggage 404, it is preferable that the vacuum be rated to draw air at the rate of 100 to 3000 liters per minute. Depending upon the size of the baggage, it may even be more preferable to extract air from the baggage at the rate of 100 to 1000 liters per minute. It is also preferable that the vacuum draw the air sample through the collector 1000 for about 5 to 10 seconds, and even more preferable to extract air from the baggage and through the collector for 15 seconds. As the air passes through the collector 1000, the constituents (i.e., particles and vapor molecules) in the air adhere to the porous material 106 of the test strip 102. Thus, the porous material 106 is a means for collecting the constituents of the air.

Referring to FIGS. 2 and 2A, after extracting a sample of air through the collector 1000, the test strip 102 is removed from the collector 1000 and inserted into a testing unit 200 capable of testing for trace amounts of explosives, such as an EGIS Explosives Detection System. It is preferred that the end of the collector 1000 that has the porous material 106 be inserted into the testing unit 200. The EGIS Explosives Detection System includes a vacuum source (not shown). Thus, the constituents are drawn from the porous material 106 of the test strip 102 and into the testing unit and analyzed. That is, by inserting the test strip 102 into a testing unit that has the capability to detect and analyze the constituents of air, such as the EGIS Explosives Detection System, and operating that testing unit, an indication of whether the constituents explosive materials will be produced. Using the process outlined above, if the testing unit indicates the test strip 102 includes explosive air-borne constituents, then the interior of the baggage may contain such explosives.

The process discussed above outlines drawing a sample of air through a collector and testing the test strip in the collector after such air sample is extracted from one piece of baggage. However, a collector must be cleaned before it can be used again. Thus, assuming each airline passenger's baggage was checked using a separate collector, the total number of collectors used to screen all of the carry-on and checked baggage for a single flight could be extraordinary. Therefore, it may be more efficient to screen groups of baggage using a single collector rather than screening each individual piece of baggage using a single collector. That is, it may be preferable to screen baggage using a batch process.

For example, assuming a baggage-collector interface 400 was already inserted into a predetermined number (i.e., a group of five, ten, fifteen, or twenty bags), a single collector 1000 could be inserted into a plurality of baggage-collector interfaces 400 corresponding to the predetermined number of baggage pieces. The method used to extract the series of air samples could include using the means illustrated in FIG. 19. Thereafter, the test strip 102 of the collector 1000 could be removed from the collector and inserted into the explosives testing unit, such as the EGIS Explosives Detection System, to determine whether any of the predetermined number of bags contain explosives. If the testing unit indicates the test strip 102 contains explosive constituents, then that would indicate the presence of explosives within at least one of the pieces of baggage within the batch of screened baggage.

Thereafter, all of the baggage within that group (e.g., batch) could be individually screened for explosives. Further screening methods could include using the method described above. That is, a separate collector would be used to screen each piece of baggage within the predetermined group, if the test strip of the collector used to screen all of the pieces of baggage yields a positive trace of an explosives. Another method for individually screening each piece of baggage may include manual inspection or X-ray.

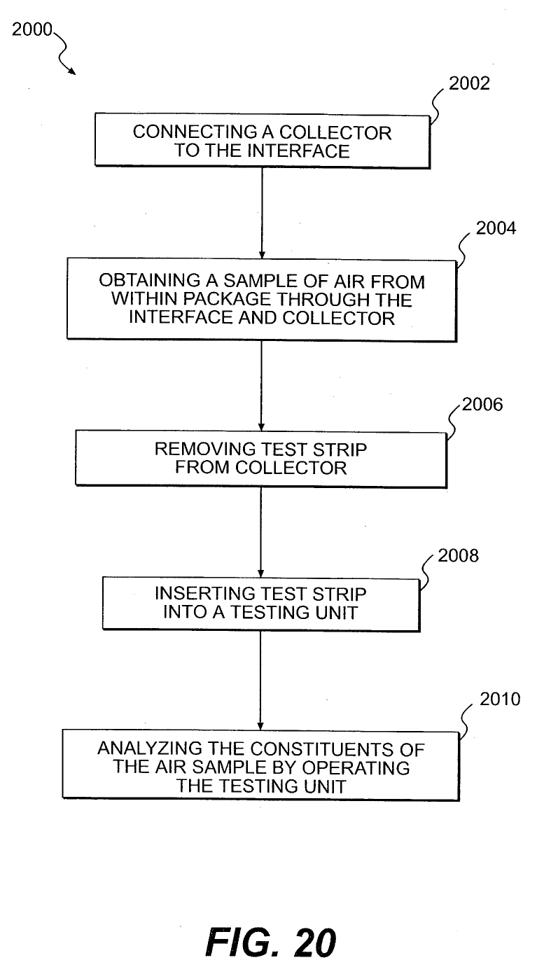

The embodiments of the present invention discussed above concern obtaining a sample of air from within a piece of baggage and analyzing the sample to determine whether the constituents of the air contain explosives. An alternate embodiment of the present invention concerns obtaining a sample of air from within a package and analyzing that sample to determine whether the constituents of that air contain explosives. Referring to FIG. 20, there is shown a flow diagram of such an embodiment. The steps of the method of this alternate embodiment include: connecting a collector, which includes a test strip, to a package-collector interface 2002; extracting a sample of air from within the package by vacuuming the air through the package-collector interface and the collector (including the test strip) 2004, thereby allowing the test strip in the collector to collect constituents of such air; removing the test strip from the collector 2006; inserting the test strip into a testing unit capable of detecting a trace amount of an explosive 2008; and analyzing the constituents of the air sample by operating the testing unit 2010.

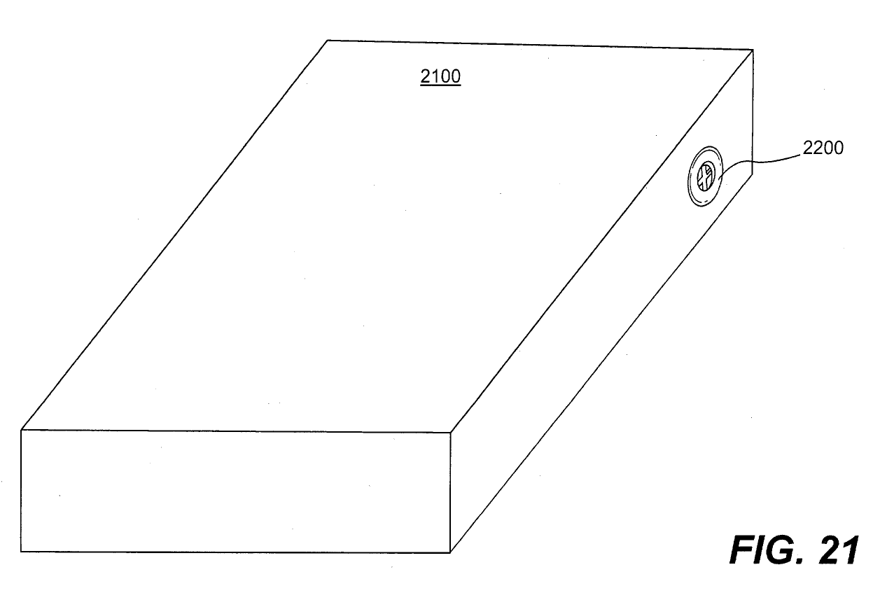

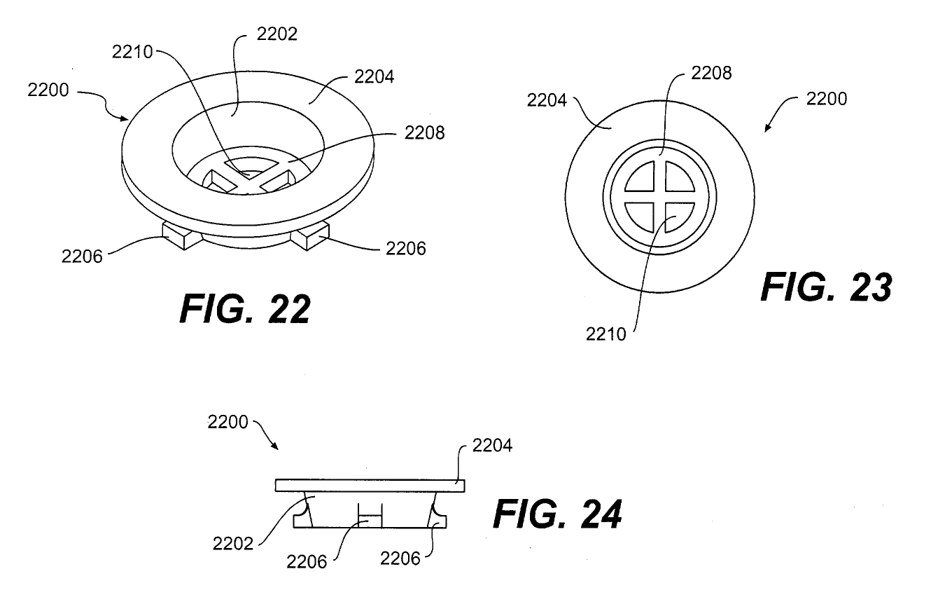

Step 2002 of connecting a collector to the package-collector interface assumes that the package-collector interface is already located within the package. Referring to FIG. 21, there is shown a package-collector interface 2200 secured within a package 2100. Referring to FIGS. 22-24, there is shown an embodiment of the package-collector interface 2200 of the present invention. The package-collector interface 2200 includes a recess created by a tubular portion 2202, a brim 2204 acting as a top securing member extending from the top of the tubular portion 2202 and a plurality of securing members 2206 extending from the bottom of the tubular portion 2202.

The interior of the tubular portion 2202 creating the recess has a shape that corresponds to the shape of the exterior of the collector 1000 discussed in detail above. Included within the recess is an abutment 2208 extending inwardly from the interior of the tubular portion 2202. The abutment 2208 provides a stopping means against which the collector 1000 rests when the collector 1000 and package-collector interface 2200 are connected. The abutment 2208, however, includes a plurality of vents 2210, thereby allowing air to pass through the package-collector interface 2200.

As mentioned above, the package collector interface 2200 includes a plurality of securing members 2206 which are located within the interior of the package 2000 when the package-collector interface is inserted therein. That is, when the package-collector interface 2200 is in the package 2000, the brim 2204 resides on the exterior of the package 2000 and the securing members 2206 on the interior of the package. It is preferred that the package-collector interface 2200 include at least a pair of securing members 2206 diametrically opposed from one another, and it is even more preferable that the package-collector interface 2200 include two pairs of securing members 2206, wherein all four securing members 2206 are evenly spaced from one another. It is also preferred that if the package-collector interface 2200 includes any other number of securing member 2206 that the securing members be evenly spaced from one another.

After the collector 1000 is connected to the package-collector interface 400, the remaining steps of FIG. 20 are performed in substantially the same manner as the corresponding steps of FIG. 3 are performed which are discussed above with respect to analyzing the constituents of air within a piece of baggage.

Although the invention has been described and illustrated with respect to the exemplary embodiments thereof, it should be understood by those skilled in the art that the foregoing and various other changes, omissions and additions may be made without departing from the spirit and scope of the invention. For example, other types of collectors-other than collectors 1000 and 1000 described herein-could be used in conjunction with the baggage-collector interface and package-collector interface. One such alternative collector is a preconcentrator discussed in application Ser. Nos. 10/224,688 and 10/224,710 assigned to the assignee of the present invention and hereby incorporated by reference.

What is claimed is:

1. A system for collecting constituents of air from within the interior of a package, said system comprising:
(a) a collection device comprising
(i) a slotted plate comprising a first side, a second side, a slot between said first and second sides, and a passageway through said slotted plate, wherein said passageway is perpendicular to said slot;
(ii) a test strip located within said slot, said test strip comprising an aperture and a porous layer overlapping at least a portion of said aperture, wherein at least a portion of said porous layer is aligned with said passageway; and
(iii) a collection-device tube extending from said second side of said slotted plate and aligned with at least a portion of said passageway; and
(b) an interface between said collection device and a package, said interface comprising
(i) an interface tube having a top end and a bottom end, said interface tube matingly engaging said collection-device tube;
(ii) a brim extending from said top end of said interface tube; and
(iii) a plurality of securing members extending from said bottom end of said interface tube.

2. The system of claim 1 wherein said plurality of securing members comprises two securing members diametrically opposed from to another.

3. The system of claim 1 wherein said plurality of securing members comprises four securing members, thereby creating two pairs of securing members, wherein said securing members within each of said pairs are diametrically opposed from to another.

4. The system of claim 1 wherein said interface further comprises an abutment extending inwardly from the interior of said interface tube at said bottom end of said interface tube, said abutment including an opening thereby allowing air to pass therethrough.

5. An interface between a collection device and a package, said interface comprising
(a) a tube having a top end, a bottom end and an interior shape, wherein said interior shape of said interface tube corresponds to an exterior shape of a portion of a collection device;
(b) a brim extending from said top end of said interface tube; and
(c) a plurality of securing members extending from said bottom end of said interface tube.

6. The interface of claim 5 wherein said plurality of securing members comprises two securing members diametrically opposed from to another.

7. The interface of claim 5 wherein said plurality of securing members comprises four securing members, thereby creating two pairs of securing members, wherein said securing members within each of said pairs are diametrically opposed from to another.

8. The interface of claim 5 wherein said interface further comprises an abutment extending inwardly from the interior of said interface tube at said bottom end of said interface tube, said abutment including an opening thereby allowing air to pass therethrough.

9. A method of collecting constituents of air from within the interior of a package, said method comprising the steps of:
(a) inserting a collection device into an interface within a package, wherein said interface comprises an interface tube having an inside end and an outside end, wherein said inside end is within said package and said outside end is outside said package, said collection device comprising:
(i) a slotted plate comprising a first side, a second side, a slot between said first and second sides, and a passageway through said slotted plate, wherein said passageway is perpendicular to said slot;
(ii) a test strip located within said slot, said test strip comprising an aperture and a porous layer overlapping at least a portion of said aperture;
(iii) a collection-device tube extending from said second side of said slotted plate and aligned with at least a portion of said passageway, said collection-device tube matingly engaging said interface tube when said collection-device tube is inserted into said interface; and
(b) extracting air from the interior of the piece of baggage through said interface tube and through at least a portion of said porous layer of said test strip, thereby allowing said porous layer to collect constituents of air as the air passes therethrough.

10. A method of testing the air within the interior of a piece of a package, said method comprising the steps of:
(a) inserting a collection device into an interface within a package, wherein said interface comprises an interface tube having an inside end and an outside end, wherein said inside end is within said package and said outside end is outside said package, said collection device comprising:
(i) a slotted plate comprising a first side, a second side, a slot between said first and second sides, and a passageway through said slotted plate, wherein said passageway is perpendicular to said slot;
(ii) a test strip located within said slot, said test strip comprising an aperture and a porous layer overlapping at least a portion of said aperture;
(iii) a collection-device tube extending from said second side of said slotted plate and aligned with at least a portion of said passageway, said collection-device tube matingly engaging said interface tube when said collection-device tube is inserted into said interface; and
(b) extracting air from the interior of the piece of baggage through said interface tube and through at least a portion of said porous layer of the test strip, thereby allowing said porous layer to collect constituents of air as the air passes therethrough;
(c) removing said test strip from said slot within said collection device; and
(d) analyzing the constituents by placing the test strip into a testing unit and operating said testing unit.

11. The method of claim 10 wherein said testing unit is capable of detecting a trace amount of an explosive and said step of analyzing the constituents comprises analyzing the constituents to determine whether the constituents include a trace amount of an explosive.

12. The method of claim 10 wherein said testing unit is capable of detecting a trace amount of a narcotic and said step of analyzing the constituents comprises analyzing the constituents to determine whether the constituents include a trace amount of a narcotic.
